(19) United States
(12) Patent Application Publication
(10) Pub. No.: US 2011/0040208 A1
(43) Pub. Date: Feb. 17, 2011

(54) INTEGRATED LANCET AND TEST STRIP AND METHODS OF MAKING AND USING SAME

(75) Inventors: Daniel R. MCMINN, Alameda, CA (US); Mark S. Yahnke, Alameda, CA (US)

Correspondence Address: Abbott Diabetes Care Inc.;Bozicevic, Field & Francis LLP, 1900 University Ave, Suite 200, East Palo Alto, CA, 94303, US

(73) Assignee: Abbott Diabetes Care Inc.

(21) Appl. No.: 12/539,483

(22) Filed: Aug. 11, 2009

(51) Int. Cl. A61B 5/151

(52) U.S. Cl. 600/583

(57) ABSTRACT

An integrated lancet and test strip for measuring an analyte level is provided. The test strip includes a protrusion at a sample receiving end of the test strip, and a notch at a body receiving end of the test strip. Further, a method of measuring an analyte level in a sample of bodily fluid using the integrated lancing and testing meter is also provided. Still further, a method of making a plurality of test strips for use in an integrated lancet and test strip is also provided.

### BACKGROUND OF THE INVENTION

The prevalence of diabetes is increasing markedly in the world. At this time, diagnosed diabetics represent about 3% of the population of the United States. It is believed that the actual number of diabetics in the United States is much higher. Diabetes can lead to numerous complications, such as, for example, retinopathy, nephropathy, and neuropathy.

The most important factor for reducing diabetes-associated complications is the maintenance of an appropriate level of glucose in the blood stream. The maintenance of the appropriate level of glucose in the blood stream may prevent and even reverse some of the effects of diabetes.

Analyte, e.g., glucose, monitoring devices known in the art have operated on the principle of taking blood from an individual by a variety of methods, such as by means of a needle or a lancet. The individual then coats a plastic strip carrying reagents with the blood, and finally inserts the blood coated strip into a blood glucose meter for measurement of glucose concentration by optical or electrochemical techniques. Or, alternatively, the plastic strip is inserted into the meter and then blood is applied to the strip.

Medical devices of the prior art have begun to use integrated lancet and test strips. The integrated lancet and test strips are formed from various layers and comprise a channel between layers. The channel can become occluded when the test strip (the sensor containing portion) contacts the sample site. Occlusion of the channel may result in less success for a reading, slower fill times, and more times necessary to re-apply the sample.

### SUMMARY OF THE INVENTION

An integrated lancet and test strip for measuring an analyte level as well as methods of making and using the same are provided. The integrated lancet and test strip includes a body, and the body includes a test strip receiving end and a lancet end. The integrated lancet and test strip also includes a lancet needle coupled to the lancet end of the body, and a test strip coupled to the test strip receiving end of the body. The test strip includes multiple electrodes and assay chemistry for determining an analyte level in a sample, a protrusion at a sample receiving end of the test strip, and a notch at a body receiving end of the test strip. The test strip and lancet needle are also configured such that the test strip is not in fluid communication with the lancet needle.

These and other objects, advantages, and features of the invention will become apparent to those persons skilled in the art upon reading the details of the embodiments as more fully described below.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures:

FIG. 1 illustrates a perspective view of an integrated lancing and testing meter and exploded perspective view of the assembly for storing and dispensing integrated lancet and test strips, according to certain embodiments.

FIG. 2 illustrates a perspective view of selected components of a lancing/collecting assembly of a medical diagnostic apparatus and integrated lancet and test strip, according to certain embodiments.

FIGS. 3A-B illustrate an exploded perspective view and perspective view, respectively, of an integrated lancet and test strip, according to certain embodiments.

FIGS. 4A-B illustrate an exploded perspective view and perspective view, respectively, of an integrated lancet and test strip, according to certain embodiments.

FIG. 5 illustrates a perspective view of a test strip, according to certain embodiments.

FIG. 6 illustrates an exploded perspective view of a test strip, according to certain embodiments.

FIGS. 7A-F illustrate top planar views of a test strip having various shapes of protrusions and notches, according to certain embodiments.

FIGS. 8A-C illustrate top planar views of a test strip having protrusions and notches in various locations, according to certain embodiments.

FIG. 9 is a flow diagram of a method of measuring an analyte level in a sample of bodily fluid using an integrated lancing and testing meter, according to certain embodiments.

FIGS. 10A-B illustrate top planar views of a layered structure being separated into test strips, according to certain embodiments.

FIGS. 11A-11B illustrate top planar views of a layered structure being separated into test strips, according to certain embodiments.

FIGS. 12A-12B illustrate top planar views of a layered structure being separated into test strips, according to certain embodiments.

FIG. 13 illustrates a flow diagram for measuring an analyte level in a sample of bodily fluid using an integrated lancing and testing meter, according to certain embodiments.

FIGS. 14A-14M illustrate in schematic form a method of measuring an analyte level in a sample of bodily fluid using an integrated lancet and testing meter, according to certain embodiments.

### DETAILED DESCRIPTION OF THE INVENTION

Before the present invention is described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “an electrode” includes a plurality of such electrodes and reference to “the electrode” includes reference to one or more electrodes and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Medical Diagnostic Device and Assembly

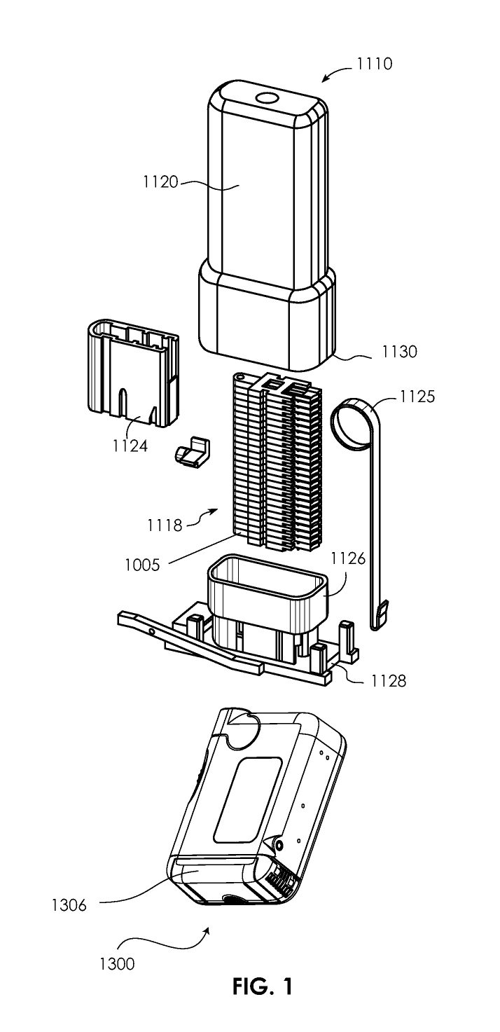

FIG. 1 illustrates a medical diagnostic device 1300 and an assembly 1110 for storing and dispensing integrated lancet and test strips (shown removed from device 1300), according to an embodiment. The example device shown is a point and shoot medical diagnostic device. By point and shoot, it is meant that a user places the meter on the skin location chosen for fluid/blood extraction, and is merely required to push a button to activate the device and then simply wait for the meter to make the measurement and report blood glucose level. Device 1300 includes a cartridge door 1306 which opens to allow assembly 1110 to be inserted and removed. Device 1300 is also referred to herein as “meter” and “integrated lancet and testing meter”.

As shown in FIG. 1, the assembly 1110 for storing and dispensing integrated lancet and test strips includes a magazine 1118 including a plurality of integrated lancet and test strips 1005, each integrated lancet and test strip comprising a lancet-containing portion and a sensor-containing portion (also referred to herein as “test strip”). Integrated lancet and test strips that are suitable for use with a medical diagnostic device (also referred to herein as an “integrated lancet and testing meter”) are provided for in the sections to follow. Detailed description of test strips, and embodiments thereof, that may be integrated with the lancet are also provided for in sections to follow. The magazine 1118 has an exterior cover 1120. The purpose of the exterior cover 1120 is to maintain the test strips in a substantially moisture-tight, air-tight condition. Materials that are suitable for forming the exterior cover 1120 include rubber and other polymeric materials.

Inside the exterior cover 1120 may be an interior cover (not shown), which contains a desiccant. The purpose of the interior cover is to provide a second barrier to maintain the integrated lancet and test strips in a substantially moisture-tight, air-tight condition. Materials that are suitable for forming the interior cover include polymeric materials impregnated with a desiccant, e.g., plastic impregnated with desiccant. The structure of the interior cover is substantially congruent with the structure of the exterior cover 1120. The desiccant absorbs moisture that evades the exterior cover 1120. Inside the interior cover 1122 is a platform 1124 for containing a biasing element 1125, e.g., a constant force spring, for urging test strips toward the location in the magazine 1118 from which test strips are fed to a lancing/collecting assembly 112 within the device 1300. Also inside the interior cover is an insert 1126 for securing the biasing element 1125. The platform 1124 can be filled with a desiccant, in order to enhance moisture resistance of the test strips stored within the assembly 1110.

Medical diagnostic device 1300 comprises subsystems and assemblies (not shown) which facilitate the operations of the device. For example, device 1300 may include in addition to the assembly 1110 for storing and dispensing test strips, a lancing/collecting assembly, an assembly for removing a protective cover from the tip of a lancet and re-attaching the protective cover to the tip of a used lancet, and an analyzer. An end cap having an opening, through which a lancet can be projected for forming an opening in the skin of a patient, and through which a sensor can be projected for collecting a sample of biological liquid emerging from the opening in the skin of the patient. Device 1300 may also comprise one or more ports (e.g., ejection port and/or a lancing and/or testing port).

A method of measuring an analyte level in a sample of bodily fluid using an integrated lancing and testing meter is provided. The method comprises piercing a lancing site on a subject with a lancet portion of an integrated lancet and test strip comprising a lancet and a test strip. The test strip comprises multiple electrodes and assay chemistry for determining the analyte level in the sample, a protrusion at a sample receiving end of the test strip, and a notch at a body receiving end of the test strip. The method further comprises automatically re-orienting the integrated lancet and test strip within the meter, and automatically advancing and contacting the integrated lancet and test strip at the lancing site to measure an analyte level in a sample of bodily fluid, the integrated lancet and test strip first contacting a protrusion at the receiving end of the test strip to the lancing site.

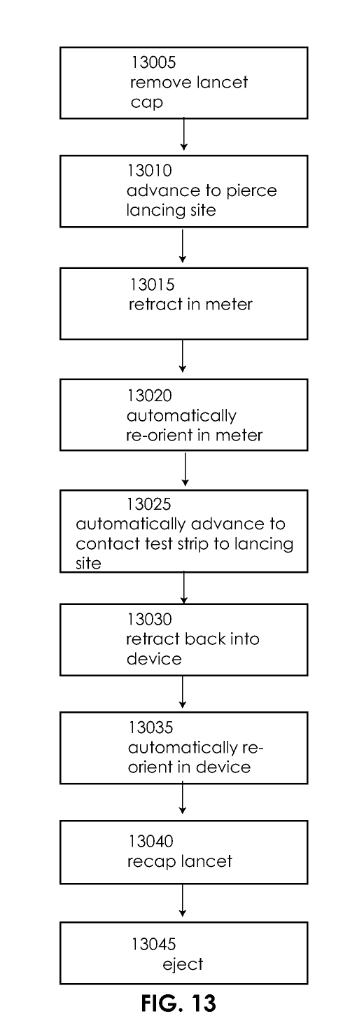

Referring for a moment to FIG. 13, a flow diagram is illustrated for measuring an analyte level in a sample of bodily fluid using an integrated lancing and testing meter, according to an embodiment. At block 13005, a lancet cap is removed from a lancet on an integrated lancet and test strip.

At block 13010, the integrated lancet and test strip is advanced to pierce a lancing site on a subject with a lancet portion of the integrated lancet and test strip. If an ejection port and separate lancing/testing port are implemented, then the lancet is advanced out the lancing/testing port. The test strip may comprise, for example, multiple electrodes and assay chemistry for determining the analyte level in the sample; a protrusion at a sample receiving end of the test strip (as described in further detail later); and, a notch at a body receiving end of the test strip (as described in further detail later). In some embodiments, the test strip does not comprise a notch at the body receiving end of the test strip.

At block 13015, the integrated lancet and test strip is automatically retracted within the meter. At block 13020, the integrated lancet and test strip are automatically re-oriented within the meter. At block 13025, the integrated lancet and test strip is automatically advanced to contact the test strip at the lancing site to measure an analyte level in a sample of the bodily fluid, the advancing of the integrated lancet and test strip first contacting the protrusion at the receiving end of the test strip to the lancing site. The integrated lancet and test strip first contacts a protrusion at the receiving end of the test strip to the lancing site. At block 13030, the integrated lancet and test strip is retracted back into the device. At block 13035, the integrated lancet and test strip is automatically re-oriented within the device. At block 13040, the lancet is re-capped for safety. And at block 13045, the integrated lancet and test strip is ejected. If an ejection port and separate lancing/testing port are implemented; and then the test strip is ejected through the ejection port.

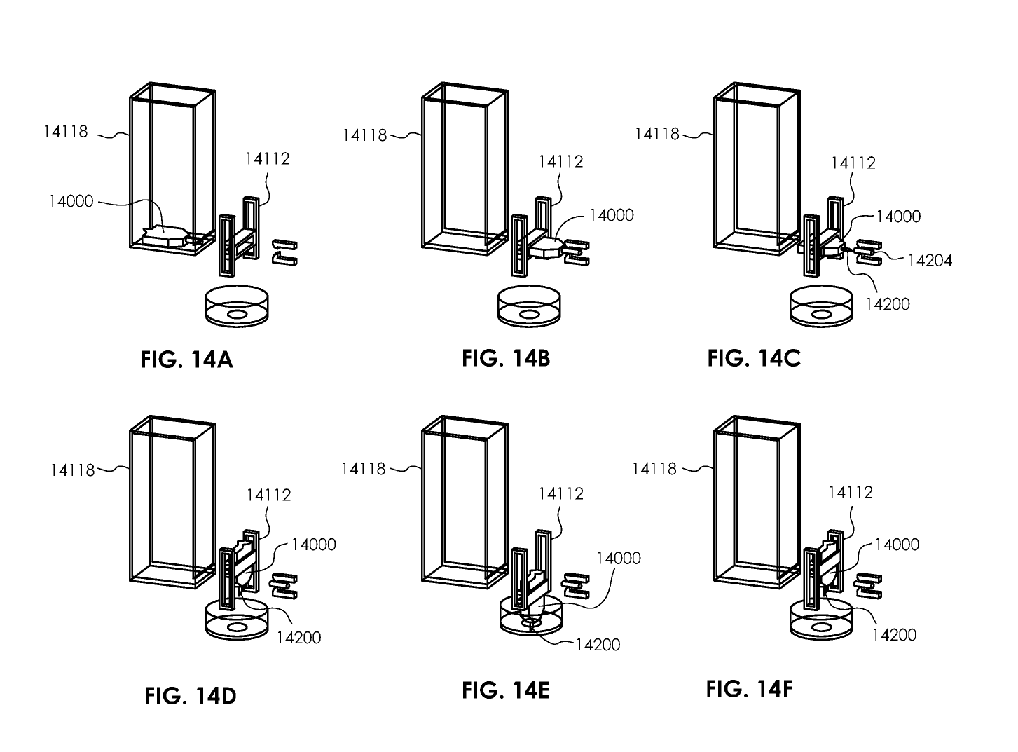

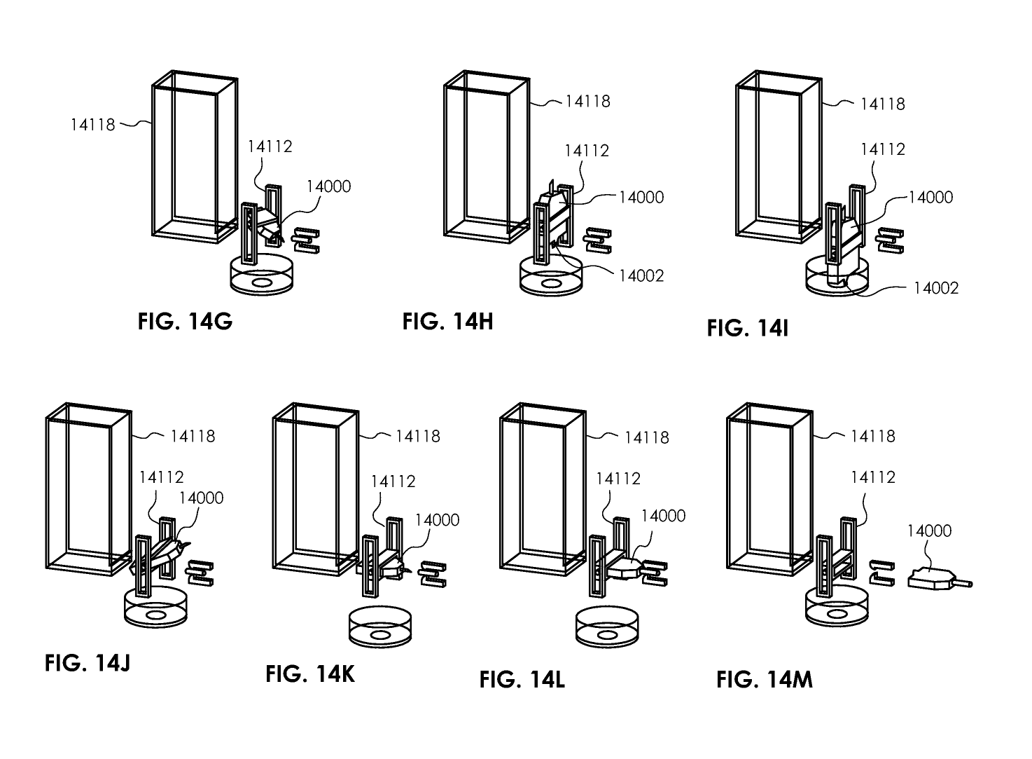

FIG. 14A through FIG. 14M, inclusive, illustrate in schematic form one way of carrying out a method of measuring an analyte level in a sample of bodily fluid using an integrated lancet and testing meter, according to embodiments herein. FIG. 14A shows an integrated lancet and test strip 14000 in the cartridge 14118. FIG. 14B shows the integrated lancet and test strip 14000 advanced from the cartridge 14118 and inserted into the lancing/collecting assembly 14112, which is represented schematically by two parallel upright elements, each element having a slot formed therein. FIG. 14C shows the protective cover 14204 being removed from the lancet 14200 of the integrated lancet and test strip 14000. It should be noted that the protective cover 14204 could be removed before the integrated lancet and test strip 14000 is inserted into the lancing/collecting assembly 14112. FIG. 14D shows the integrated lancet and test strip 14000 rotated 90° so that the lancet 14200 is in position for lancing the skin of the patient. FIG. 14E shows that the lancet 14200 has entered the skin of the patient. FIG. 14F shows that the lancet 14200 has been retracted from the skin of the patient. FIG. 14G shows that the integrated lancet and test strip 14000 is being rotated 180° so that the test strip 14002 can collect biological liquid emerging from the opening formed in the skin of the patient. FIG. 14H shows that the test strip 14002 of the integrated lancet and test strip 14000 is ready to be indexed so that the test strip 14002 can collect biological liquid emerging from the opening formed in the skin of the patient. FIG. 14I shows the test strip 14002 of the integrated lancet and test strip 14000 contacting the biological liquid emerging from the skin of the patient, first contacting a protrusion at the receiving end of the test strip to the lancing site. FIG. 14J shows that the integrated lancet and test strip 14000 is being rotated 90° so that the integrated lancet and test strip 14000 will come into the proper in position for being ejected from the medical diagnostic device. FIG. 14K shows the integrated lancet and test strip 14000 in position for ejection from the medical diagnostic device. FIG. 14L shows the protective cover 14204 being reattached to the lancet 14200. FIG. 14M shows the integrated lancet and test strip 14000 being ejected from the medical diagnostic device.

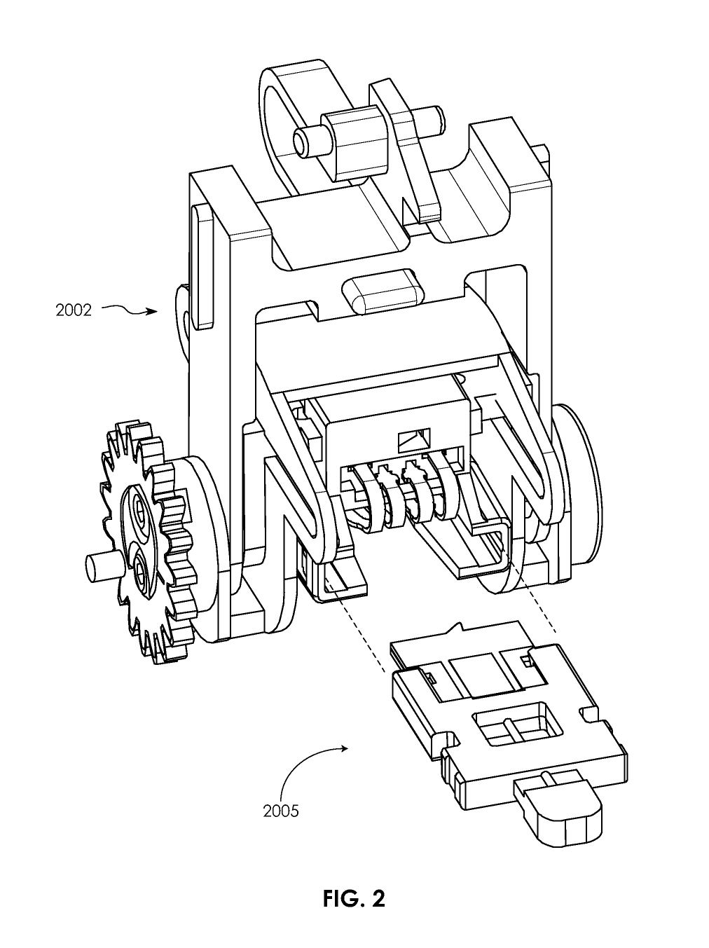

Referring to FIG. 2, a turret and integrated lancet and test strip are illustrated, according to an embodiment. Turret 2002 is a sub-assembly within device 1300 and works in conjunction with other components (not shown) within device 1300 to re-orient and advance the integrated lancet and test strip 2005 during use. For example, in an embodiment, turret 2002 receives the integrated lancet and test strip 2005; automatically re-orients the integrated lancet and test strip 2005 for piercing a lancing site; automatically advances and contacts the lancing tip at the lancing site; automatically re-orients the integrated lancet and test strip 2005 within device 1300; automatically advances and contacts the test strip at the lancing site, first contacting the protrusion at the receiving end of the test strip to the lancing site; and automatically re-orients the integrated lancet and test strip 2005 for re-capping and removal from device 1300.

Details regarding device 1300, its subsystems and assemblies, and operations thereof, are described in U.S. patent application Ser. Nos. 11/535,985, 11/535,986, 12/488,181, the entireties of which are incorporated herein by reference.

Integrated Lancet and Test Strip

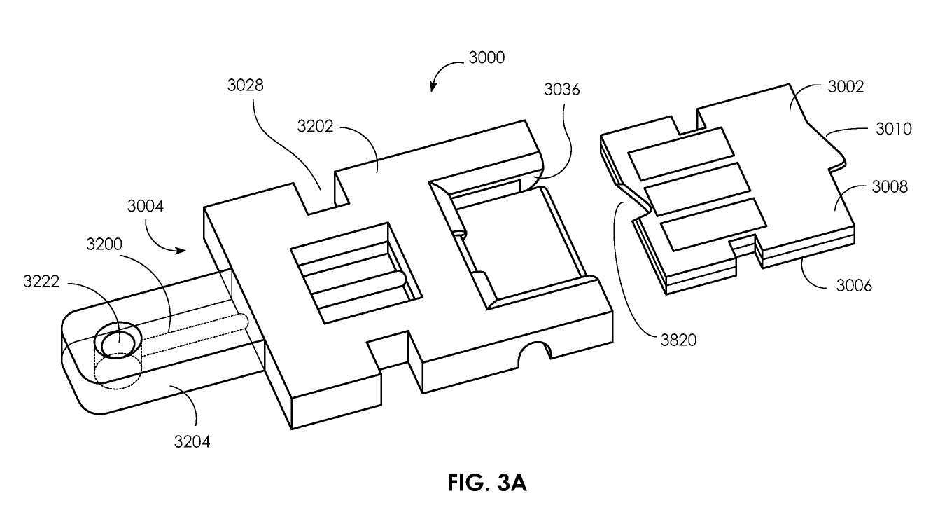

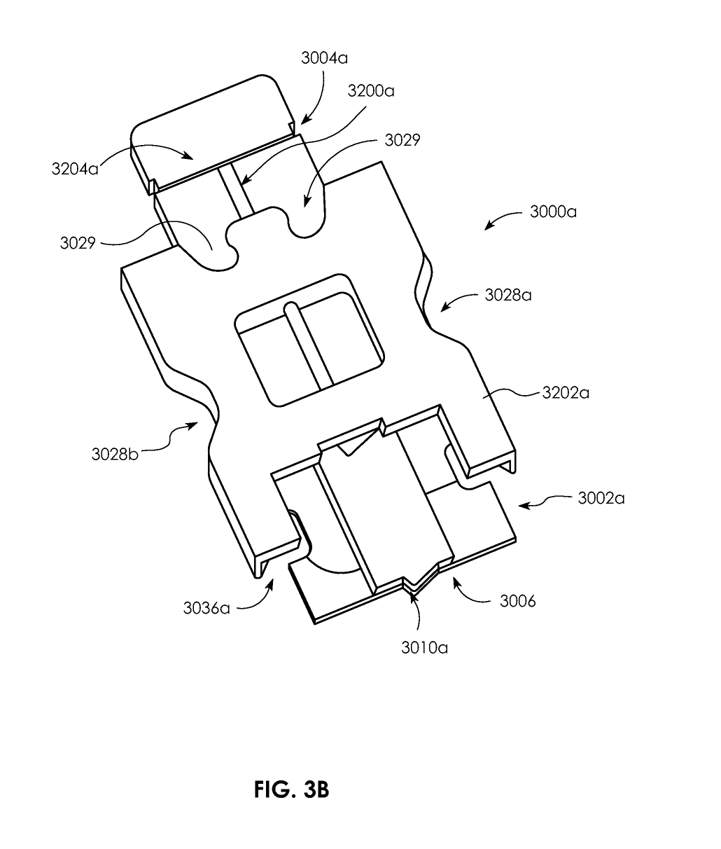

As shown in FIGS. 3A-3B, integrated lancet and test strip 3000 has a sensor-containing portion 3002 and a lancet-containing portion 3004. Referring specifically to FIGS. 3A-3B, an integrated lancet and testing 3000 is provided for measuring a body analyte, e.g., glucose, level in a diabetes care regimen. A lancet body 3202 includes a test strip receiving end 3036 and a lancet end. A lancet needle 3200 is coupled with and protruding from the lancet end and secured by a lancet cap 3204. A test strip 3002 is coupled to the test strip receiving end 3036 of the lancet body 3202 having multiple electrodes and assay chemistry for testing an analyte, e.g., glucose, level of an applied body fluid. The test strip comprises at least one protrusion on a sample receiving end. The test strip may further comprise at least one notch at a body receiving end of the test strip. The test strip 3002 and lancet 3200 are relatively disposed at different ends of the integrated lancet and test strip 3000 for providing both lancing and application of body fluid at a lancing site by reorienting and advancing the integrated lancet and test strip 3000 within the meter 1300 after lancing to contact a sample receiving portion of the test strip precisely at the lancing site. In some embodiments, the test strip and lancet needle are configured such that the test strip is not in fluid communication with the lancet needle.

The reorienting may include rotating the integrated lancet and test strip 3000 when the lancing site remains approximately at the predetermined location relative to the meter for application of body fluid to the sample receiving portion of the test strip 3002. The test strip 3002 and lancet 3200 may be symmetrically disposed at opposite ends of the lancet body 3202. The reorienting may include rotating and/or flipping the integrated lancet and test strip 3000 when the lancing site remains approximately at the predetermined location relative to the meter for application of body fluid to the sample receiving portion 3010a of the test strip 3002.

The lancet body 3202 may include a pair of relatively disposed recesses 3028a, 3028b for respectively positioning the test strip. For example, a spring-loaded ball and detent mechanism (not shown) may be implemented for lancing and application of body fluid at a same lancing/testing site. The recesses 3028a, 3028b may be a variety of shapes—e.g., rectangular, semi-circular, trapezoidal (as shown in FIG. 3B), irregular, etc.

The lancet cap 3204 of FIG. 3B includes two elastomeric arms 3029, although there may be one or more than two, that couple with defined cutouts in the lancet body 3202 for snapping the cap 3204 into and out of mating relationship with the lancet body 3202 by respective application of sufficient coupling and separation force.

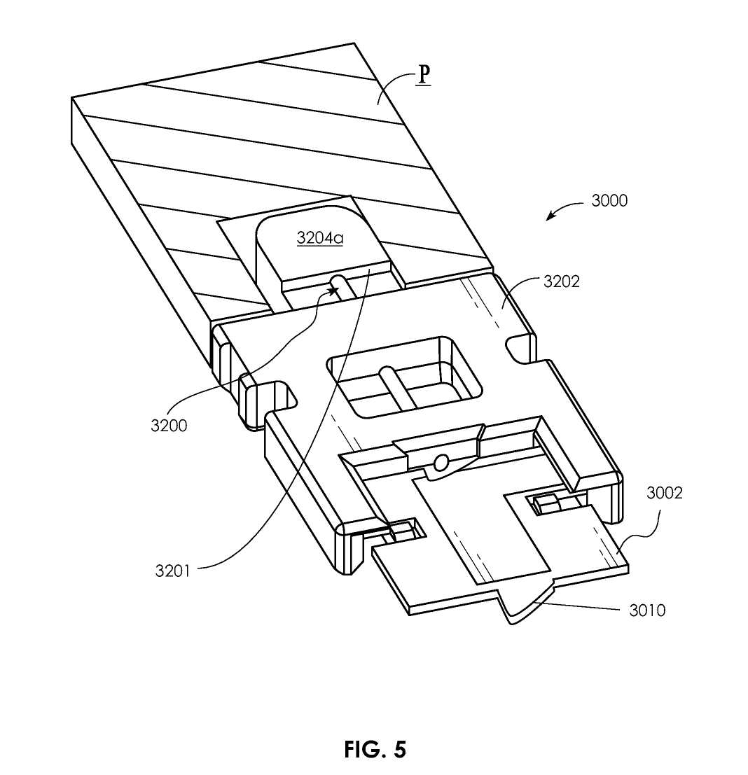

Referring for a moment to FIG. 5, an integrated lancet and test strip 3000 is shown including a lancet body 3202, test strip 3002 coupled with the lancet body 3202, and a lancet cap 3204 protecting a lancet 3200 which is also coupled to the lancet body 3202. Meter 1300 may include a pusher P, which pushes the integrated lancet and test strip 3000 into a turret (e.g., turret 2002 shown in FIG. 2). Pusher P is shown in FIG. 5 coupled with the integrated lancet and test strip 3000, and having a U-shape. Pusher P, however, may have any of a variety of shapes that fit somewhat snugly such as to overlap the lancet cap 3204 at least through the plane of a mating contour 3201 of the lancet cap 3204. Although not shown in FIG. 5, the pusher may have a corresponding contour to the mating contour 3201 of the lancet cap 3204. Meter 1300 may also include a blade or decapping lever that moves down to engage the lancet cap 3204 and uncap it from the integrated lancet and test strip 3000. When the blade is disposed in mating relation with the mating contour 3201 of the lancet cap 3204, the pusher P is also coupled, via its own corresponding contour or sufficient friction, with the blade and/or with the lancet cap 3204. This permits a retreating motion of the pusher P to bring the lancet cap 3204 with it away from the lancet body 3202 of the integrated lancet and test strip 3000 for arming the lancet 3200 while the integrated lancet and test strip 3000 is disposed in the turret 2002 shown in FIG. 2. Although not shown, a chain or other flexible component may be attached to the pusher P for advancing and retreating the pusher P.

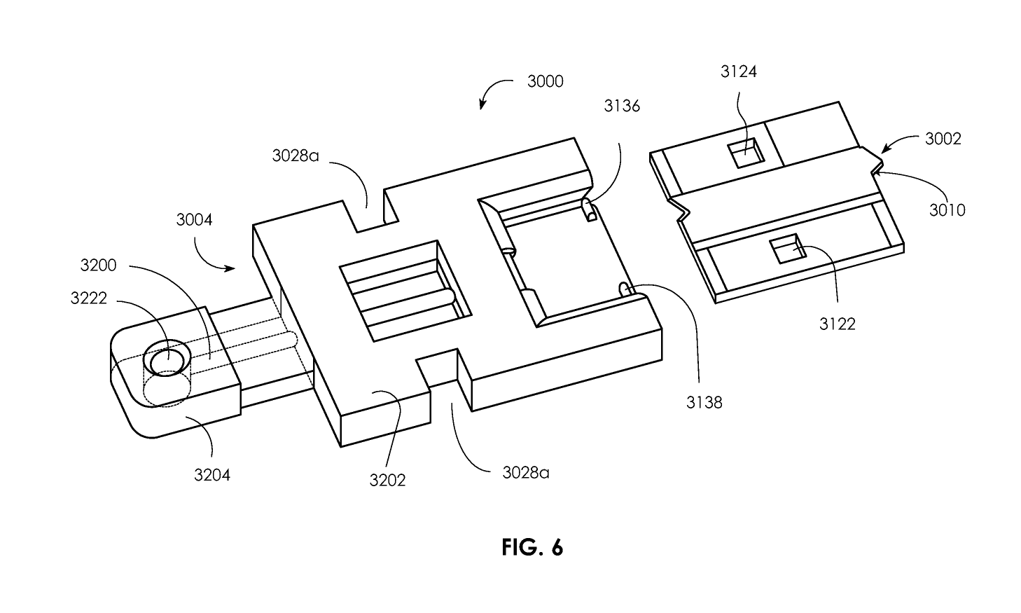

FIG. 6 illustrates an exploded perspective view of an integrated lancet and test strip, according to another embodiment. Integrated lancet and test strip 3000 is shown including a lancet body 3202, test strip 3002 coupled with the lancet body 3202, and a lancet cap 3204 protecting a lancet 3200 which is also coupled to the lancet body 3202. Shown in this embodiment, integrated lancet and test strip 3000 further comprises at least two teeth 3136, 3138 in lancet body 3202; and, corresponding slots 3122, 3124 in test strip 3002 for coupling the lancet body 3202 and test strip 3002 together.

Test Strips

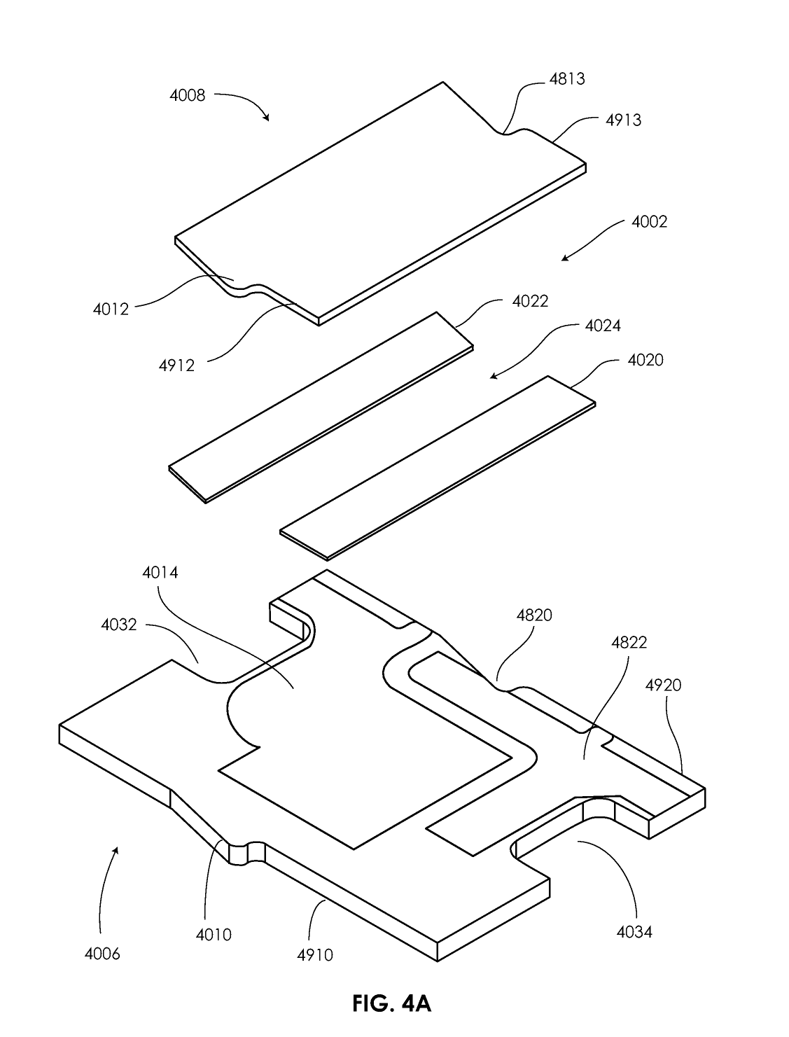

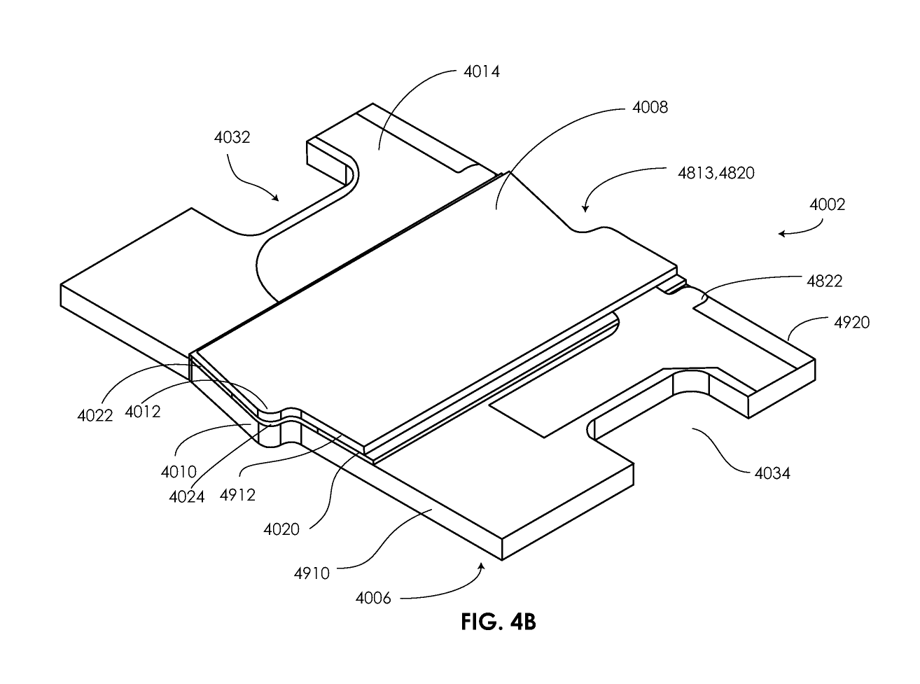

FIGS. 4A-B illustrate test strips which can be integrated within an integrated lancet and test strip, according to certain embodiments. FIG. 4A is an exploded perspective view of a test strip, according to an embodiment; and, FIG. 4B is a perspective view of a test strip, according to another embodiment.

Test strip 4002 is shown comprising a base 4006 and a cover 4008. As shown in FIGS. 4A-B, both the base 4006 and the cover 4008 are substantially rectangular in shape, although other shapes may be used. In this substantially rectangular embodiment, base 4006 has two major surfaces and four edges. Cover 4008 also has two major surfaces and four edges. Base 4006 has a protrusion 4010 formed in one edge 4910 thereof, and the cover 4008 has a protrusion 4012 formed in one edge 4912 thereof. Base 4006 has a notch 4820 formed in one edge 4920 thereof, and the cover 4008 has a notch 4813 formed in one edge 4913 thereof. In FIGS. 4A-B, protrusions 4010 and 4012 are located at an edges 4910 and 4912, respectively, that forms the sample receiving portion of the test strip, and notches 4813 and 4820 are shown at an edges 4913 and 4920, respectively, opposite the edges of the protrusion. However, the notches may be at a different edge depending on design. The term notch is used herein interchangeably with recess, and can similarly be referred to as a groove, indentation, etc. When integrated to a lancet, the lancet may be positioned approximately 180° from the protrusions 4010 and 4012.

The surfaces of these protrusions 4010 and 4012 may bear a hydrophilic material in order to enable the sample of biological liquid to have greater affinity for the protrusions 4010 and 4012 than if the protrusions were not bearing a hydrophilic material. The base 4006 may be made from an electrically non-conducting material, e.g., an insulating material that is not capable of carrying substantial electric charge or current. Examples of materials usable include polyesters, polyethylene (both high density and low density), polyethylene terephthalate, polycarbonate, vinyls, and the like. The material may be treated with a primer or other such coating to improve the adhesion of the electrodes thereon. In certain embodiments, the base and/or cover is made from a hydrophobic polymeric material, e.g., “MELINEX” polymer, or the like. Cover 4008 is made from an electrically conducting material. In certain alternative embodiments, cover 4008 is made from an electrically non-conducting material such as those described above for base 4006.

On the major surface of the base 4006 facing the cover 4008 is a layer of electrically conductive material 4822 in a first area and a layer of electrically conductive material 4014 in a second area. The first area constitutes the fill indication electrode and the second area constitutes the working electrode. Conductive material that may be used include gold, carbon, platinum, ruthenium dioxide, palladium, and conductive epoxies, such as, for example, ECCOCOAT CT5079-3 Carbon-Filled Conductive Epoxy Coating (available from W. R. Grace Company, Woburn, Mass.), Ag/AgCl, Ag/AgBr, as well as other materials known to those skilled in the art.

The cover 4008 is spaced from the base 4006 by layers 4022, 4020 of non-conductive adhesive in such a manner that a channel 4024 forming a sample flow path. This channel 4024 runs along the center of the test strip 4002. The non-conductive adhesive 4020 in a first area and layer of non-conductive adhesive 4022 in a second area also bond the cover 4008 to the base 4006. The cover 4008 is made of an electrically conductive material (such as, for example, vinyl having an electrically conductive material, e.g., Ag/AgCl, thereon) and functions as a dual purpose reference/counter electrode. When a sample of biological liquid is introduced at the hydrophilic protrusion (i.e., protrusions 4010 and 4012), the sample is easily drawn up into the channel 4024, along which the sample flows by means of capillary attraction. The protrusion in the sample receiving end contributes to breaking the surface tension of the fluid sample and to assist it in filling faster. Portions of the electrically conductive material of the base 4006 function as electrical contact pads.

While not critical, it is advantageous that the dimensions of the test strip 4002 be as small as possible in order to reduce the size of the assembly 1110 and reduce the volume of sample required to carry out a test. Typical dimensions of the base 4006 and cover 4008 are approximately 6 mm×6 mm x<2 mm. Typical dimensions of the electrodes and typical dimensions of a sample flow channel 4024 are described in U.S. Pat. Nos. 6,229,757 and 6,616,819, incorporated herein by reference. When the sample of biological liquid is introduced at the hydrophilic protrusions 4010, 4012, the liquid is easily drawn up into the channel 4024, along which the liquid flows by means of capillary attraction.

The base 4006 is shown having two recesses 4032, 4034 in the edges perpendicular to the edge having the sample uptake protrusion 4012. The function of these recesses 4032, 4034 in the sides is to securely attach the test strip 4002 to the lancet-containing portion 3004 of the integrated lancet and test strip 3000, which holds the lancet in place. For example, tabs from the lancet-containing portion 3004 of the test strip 3000 may project downward toward the recesses 4032, 4034 in the edges of the test strip 4002. In an embodiment, the test strip 4002 does not have two recesses, but rather, at least two or more openings in the base. In this case, the tabs of the lancet-containing portion of the test strip may project upwardly. It should be understood that the tabs may be designed to project downwards or upwards whether using the recesses or openings.

Below a sample application well or zone of a test strip may be a wicking membrane that is striped with various reagents to create various reagent, capture and/or eluate zones. A hemolysis reagent zone may be positioned below a sample application zone. The hemolysis reagent zone may include a hemolysis reagent that is striped, such as absorbed, confined, or immobilized, on a wicking membrane of the test strip. A small amount of hemolysis reagent, such as about 1 to about 2 or about 3 microliters, for example, is sufficient for striping the wicking membrane such that the hemolysis reagent zone is sufficiently confined on the test strip. Any reagent or combination of reagents suitable for hemolysis, and the consequent liberation of hemoglobin, can be used. By way of example, an ionic detergent, such as sodium dodecyl sulfate (SDS), a non-ionic detergent, such as a octylphenol ethylene oxide condensate or octoxynol-9 or t-octylphenoxypolyethoxy-ethanol, sold under the name, Triton X-100, and commercially available from Sigma Chemical or Sigma-Aldrich Co., or a hypotonic solution, may be used as a hemolysis reagent.

A glycated hemoglogin capture zone may be disposed downstream relative to the hemolysis zone. By way of example, any chemical reagent comprising at least one boron ligand, such as phenyl boronate or other boron affinity chemistry used in the above-referenced Glycosal test, or such as m-aminophenylboronic acid, such as that of a gel that is immobilized on cross-linked, beaded agarose, any antibody, such as anti-HbA1c antibody available from a number of sources, any immunoassay reagent, any chemical reagent including at least one binding ligand, such a boronic acid involving boron binding ligands, and the like, and any combination thereof, that is suitable for the binding of glycated hemoglobin to a capture zone, such as via covalent bonds, for example, or the capture of glycated hemoglobin in capture zone, may be used. A hemolysis layer/zone and a glycated hemoglobin capture zone can be integrated to form an integrated reagent zone.

A lancet can be integrated directly into the test strip 4002. Alternatively, the lancet can be attached to the lancet-containing portion of the integrated lancet and test strip. The medical diagnostic device 1300 can have an alignment feature to ensure that movement, e.g., rotation, of the test strip during use does not result in misalignment of the sample application zone of the test strip. The alignment feature can be provided by springs associated with a carrier.

The lancet-containing portion shown in FIGS. 3A-B and 5-6 can be used with, or can be modified to be used with, any of the test strips described herein. For example, the tabs for connecting the lancet-containing portion to the sensor-containing portion can be modified to project upwardly to enable the lancet-containing portion to be used with a sensor-containing portion having openings in the base, rather than recesses in the sides of the base and/or the cover. It should be noted that other embodiments of the lancet-containing portion can be used with any of the test strips described herein. As shown in FIGS. 3A-B and 5-6, the lancet-containing portion 3004 is shown as having a lancet-containing body 3202. The lancet 3200 is held in the lancet-containing body 3202, and the lancet-containing body 3202 can be attached to the test strip by tabs. Any suitable dimensions of the lancet-containing body may be employed, and in certain embodiments the lancet-containing body 3202 of the lancet-containing portion 3004 is 10 mm×8 mm×1.5 mm. Typical dimensions of the protective cover 3204 for the lancet 3200 are 3 mm×1.4 mm. Typical dimensions of the needle for forming the lancet 3200 are 28 to 30 gauge, 10 mm total length, 3.5 mm exposed length.

It should be noted that all of the embodiments of the integrated lancet and test strip shown herein are characterized by having the tip 3222 of the lancet 3200 of the lancet-containing portion 3004 of the integrated lancet and test strip located 180° from the uptake protrusion 3010 of the test strip 3002. Such positioning renders the test strips suitable for use with the medical diagnostic device.

The test strips and the magazines 1118 containing a plurality of integrated lancet and test strips can be made by the following process: To prepare the lancet-containing portion 3004 of a test strip, unfinished lancets are provided. These unfinished lancets are ground and cut to 10 mm. The ground, cut lancets 3200 are then molded into a plastic body 3202 to form the lancet-containing portion 3004 of the test strip. To prepare the sensor-containing portion 3002 of the test strip, the electrodes are printed onto the backing or cover, the appropriate reagents are coated over the electrodes, and the cards of sensor-containing portions 3002 are singulated to form individual sensor-containing portions 3002. The individual sensor-containing portions 3002 are combined with the lancet-containing portions 3004 to form completed test strips. Pluralities of test strips are then loaded into magazines 118.

The test strips described herein may be configured for analysis of an analyte in a small volume of sample by, for example, coulometry, amperometry, and/or potentiometry. The test strips may also be configured for optical analysis. The test strips may be configured to determine analyte concentration in about 1 μL or less of sample, e.g., 0.5 μL or less of sample e.g., 0.25 μL or less of sample e.g., 0.1 μL or less of sample. The chemistry of the test strips generally includes an electron transfer agent that facilitates the transfer of electrons to or from the analyte. One example of a suitable electron transfer agent is an enzyme which catalyzes a reaction of the analyte. For example, glucose responsive enzymes, including glucose oxidase or glucose dehydrogenase, such as pyrroloquinoline quinone glucose, dehydrogenase (PQQ), may be used when the analyte is glucose. Other enzymes may be used for other analytes, such as hydroxybuterate dehydrogenase, a ketone responsive enzyme for detection of ketone bodies. Additionally to or alternatively to the electron transfer agent, may be a redox mediator. Certain embodiments use a redox mediator that is a transition metal compound or complex. Examples of suitable transition metal compounds or complexes include osmium, ruthenium, iron, and cobalt compounds or complexes. In these complexes, the transition metal is coordinatively bound to one or more ligands, which are typically mono-, di-, tri-, or tetradentate. The redox mediator may be a polymeric redox mediator or a redox polymer (i.e., a polymer having one or more redox species). Examples of suitable redox mediators and redox polymers are disclosed in U.S. Pat. Nos. 6,676,816, 7,074,308, 7,465,795, 6,338,790; 6,229,757; 6,605,200 and 6,605,201, which are incorporated by reference.

The test strip also includes a sample chamber to hold the sample in electrolytic contact with the working electrode. In certain embodiments, the sample chamber may be sized to contain no more than about 1 μL of sample, e.g., no more than about 0.5 μL, e.g., no more than about 0.25 μL, e.g., no more than about 0.1 μL of sample.

The magazines 1118 can be prepared by first molding the desiccants into platforms. Resilient biasing elements and the platforms are then assembled into the housings of the magazines. The magazines are then packed and shipped.

Additional aspects of analyte test strips are disclosed in U.S. patent application Ser. No. 11/461,725, filed Aug. 1, 2006; U.S. Patent Application Publication No. 2007/0095661; U.S. Patent Application Publication No. 2006/0091006; U.S. Patent Application Publication No. 2006/0025662; U.S. Patent Application Publication No. 2008/0267823; U.S. Patent Application Publication No. 2007/0108048; U.S. Patent Application Publication No. 2008/0102441; U.S. Patent Application Publication No. 2008/0066305; U.S. Patent Application Publication No. 2007/0199818; U.S. Patent Application Publication No. 2008/0148873; U.S. Patent Application Publication No. 2007/0068807; Patent Application Publication No. 2008/0119709; U.S. Pat. No. 6,616,819; U.S. Pat. No. 6,143,164; and U.S. Pat. No. 6,592,745; U.S. Pat. No. 6,071,391; U.S. Pat. No. 6,503,381; U.S. Pat. No. 6,616,819 and U.S. Pat. No. 6,893,545; the disclosures of each of which are incorporated by reference herein.

Shapes of Protrusions & Notches

As pointed out earlier, the test strip includes a protrusion at a sample receiving end and a notch at a body receiving end of the test strip. Having a protrusion at the sample receiving end helps to break surface tension of the sample and provide for a faster fill. It also provides an air gap to avoid occlusion of the capillary channel by skin when the strip is positioned for filling. When part of the protrusion extends over the edge of the channel, or when the protrusion is located near the structure, the structure that is defining the edge of the channel provides structural rigidity to the protrusion, and thus reduces potential deflection of the soft conductive top layer material. The closer the protrusion is to the supporting structure defining the edge of the channel, the more rigidity provided to the protrusion. The protrusion also provides for a tactile edge to assist in identification of where to apply blood if strip re-application is required. Having a notch in the test strip so that at least part of the notch lies within the channel, provides for a vent-like feature which helps draw blood into the sample measurement zone, by for example, capillary action.

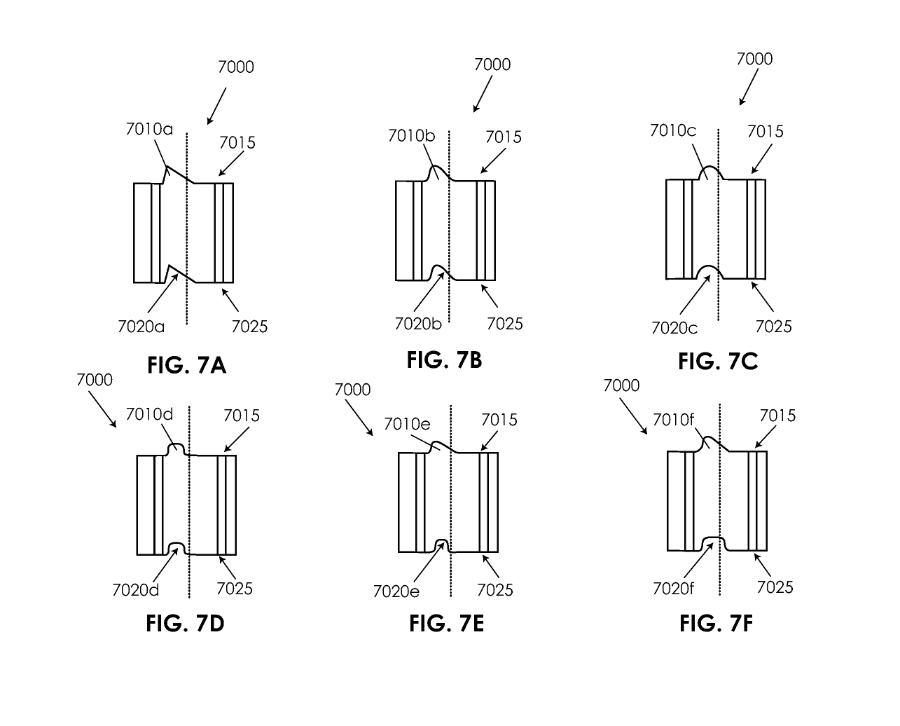

FIGS. 7A-D illustrate certain embodiments of the test strip having a protrusion corresponding to a notch. As shown in FIGS. 7A-D, test strip 7000 includes a protrusion 7010a, 7010b, 7010c, 7010d, respectively, on a sample receiving end 7015 of test strip 7000; and, a notch 7020a, 7020b, 7020c, 7020d, respectively, on a body receiving end 7025 of test strip 7000. In each figure, the shape of the protrusion corresponds to the shape of the notch—i.e., the shapes of the protrusion and notch are substantially the same.

For example, in FIG. 7A, the shape of protrusion 7010a is substantially the same as the shape of notch 7020a, with both being substantially triangular. It should be understood that substantially triangular is used broadly, and is meant to include obtuse triangles, acute triangles, any three sided polygon shape, etc., all of which are not required to be perfectly defined (e.g., lines may not necessarily be perfectly straight, corners may not necessarily be perfectly pointed, etc.). For instance, in FIG. 7B, the shape of protrusion 7010b is substantially the same as the shape of notch 7020b, with both being substantially triangular. FIG. 7C illustrates the shape of protrusion 7010c substantially the same as the shape of notch 7020c, with both being substantially semi-circular. It should be understood that substantially semi-circular is used broadly, and is meant to include oval shapes, other rounded shapes, etc., all of which are not required to be perfectly defined. In FIG. 7D, the shape of protrusion 7010d is substantially the same as the shape of notch 7020d, with both being substantially rectangular. It should be understood that substantially rectangular is used broadly, and is meant to include square shapes, other parallelogram shapes, etc., all of which are not required to be perfectly defined.

Furthermore, it should be understood that the shape of a protrusion and notch on a test strip may not be identical, but rather only need to be substantially the same. For example, if in manufacturing the protrusion and the notch, the same blade is used to make both protrusion and the notch, then the shape of the protrusion and notch are substantially the same. Or, as another example, if a protrusion is made using first blade having a substantially triangular shape, and a notch is made using a second blade having substantially triangular shape (although not the same as the first blade), then the shape of the protrusion and notch are substantially the same.

FIGS. 7E-F illustrate certain embodiment of a test strip having a protrusion that has a shape that is different than the shape of a notch. As shown in FIGS. 7E-F, test strip 7000 includes a protrusion 7010e, 7010f, respectively, on a sample receiving end 7015 of test strip 7000; and, a notch 7020e, 7020f, respectively, on a body receiving end 7025 of test strip 7000. In each figure, the shape of the protrusion is different than the shape of the notch. For example, in FIG. 7E, the shape of protrusion 7010e is different than the shape of notch 7020e, with the shape of protrusion 7010e being substantially triangular and the shape of notch 7020e being substantially semi-circular. As another example, in FIG. 7F, the shape of protrusion 7010f is different than the shape of notch 7020f, with the shape of protrusion 7010f being substantially triangular and the shape of notch 7020f being substantially rectangular.

It should be understood that the shape of the protrusion and notch may include any other shape, regular or irregular, and the combinations of shapes are not limited to the specific embodiments shown in FIGS. 7A-F. Furthermore, it should be understood that the protrusions and notches may each be of a variety of sizes.

Locations of the Protrusion and Notch

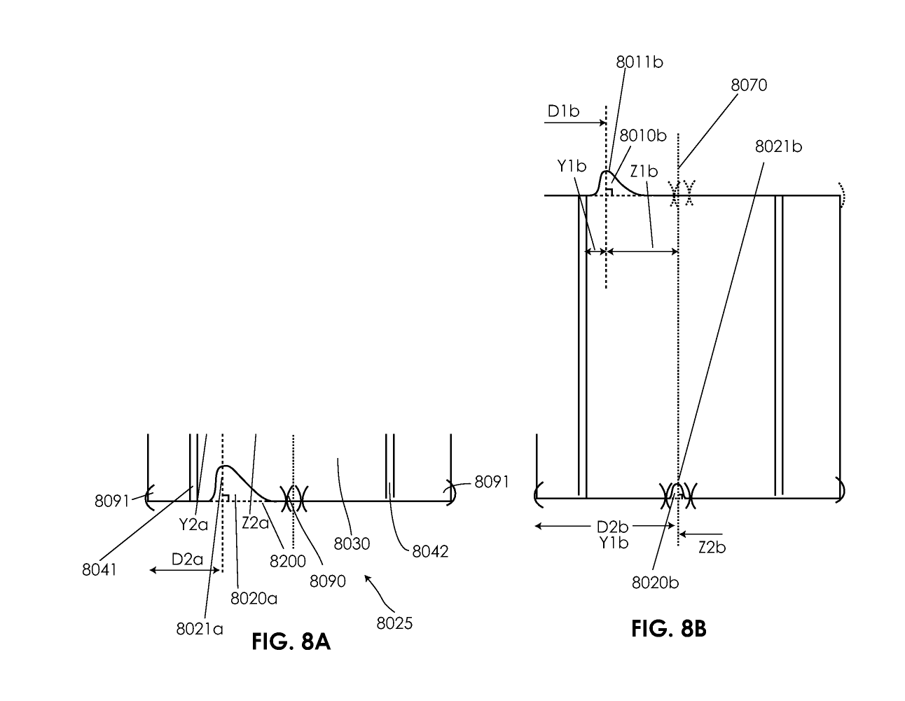

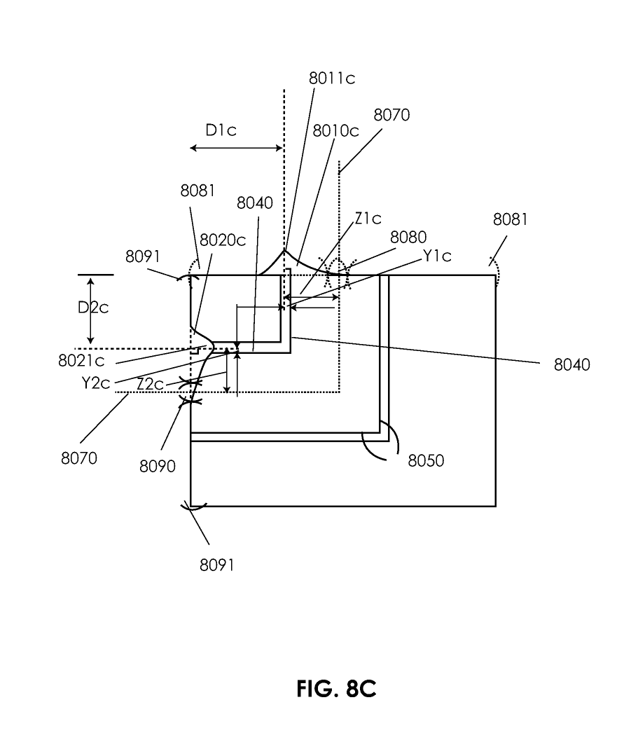

The locations of the protrusion and notch on a test strip may vary. The location of the protrusion along a sample receiving end can vary, as well as, the location of the notch along a body receiving end. FIGS. 8A-C illustrate different embodiments of test strips having a protrusion and notch in various locations along the sample receiving end and body receiving end, respectively.

It should be understood that for FIGS. 8A-C, a body receiving end may be any end of the test strip that enters the lancet body and which the channel extends to. In the specific embodiments shown, a rectangular test strip is used and thus has four edges or ends. Referring back to the embodiments shown in FIGS. 3A-D, the three edges that do not include the protrusion enter the lancet body to various degrees, with the end opposite the sample receiving end entering the most. Because the channel typically runs from the sample receiving end to the opposite end of the test strip, the body receiving end will be the end opposite the sample receiving end. However, in some embodiments, the channel may be designed to run from the sample receiving end to an end of the test strip which enters the lancet body but that is not the opposite end of the test strip. FIG. 8C illustrates such an example embodiment and is described in the corresponding description below.

In FIGS. 8A-C, test strip 8000 is shown having a protrusion 8010a, 8010b, 8010c, respectively, along a sample receiving end 8015, and a notch 8020a, 8020b, 8020c, respectively, along a body receiving end 8025, respectively.

Each protrusion 8010a, 8010b, 8010c has a peak point 8011, which is a point on the protrusion that is the furthest distance perpendicular to a base 8100 of the protrusion. Each notch 8020a, 8020b, 8020c has a peak point 8021a-c, respectively, which is a point on the notch that is the furthest distance perpendicular to a base 8200 of the notch. Channel 8030 is shown extending from sample receiving end 8015 to body receiving end 8025 of test strip 8000, and shown having a width 8060 from a first side 8040 of channel 8030 to a second side 8050 of channel 8030. Supporting structures 8041 and 8051 are shown defining sides 8040 and 8050, respectively Support structures 8041 and 8042 may be a variety of materials (e.g., non-conductive adhesive strips) used to space the cover from the base and define the channel of the test strip, as described earlier.

Bisecting line 8070 is shown bisecting channel 8030 and extending beyond the test strip 8000. Center regions 8080 and 8090 (represented by parenthesis) are the regions of sample receiving end 8015 and body receiving end 8025, respectively, near bisecting line 8070. The center region may extend a maximum of halfway to the side of the channel on each side of bisecting line 8070. Side regions 8081 and 8091 (represented by parenthesis) are the regions of sample receiving end 8015 and body receiving end 8025, respectively, outside of the respective center region.

Each protrusion and notch of FIGS. 8A-C are located a distance D1 and D2, respectively, along a sample receiving end 8015 and body receiving end 8025, respectively. As shown, distance D1 and D2 are measured from their peak point to the side of the test strip-—e.g., protrusion 8010a a distance D1a; notch 8020a a distance D2a; protrusion 8010b a distance D1b; notch 8020b a distance D2b; protrusion 8010c a distance D1c; notch 8020c a distance D2c.

In some embodiments, distance D1 and distance D2 of test strip 8000 are approximately equal. In such cases, the location of the protrusion along the sample receiving end is said to correspond to the location of the notch along the body receiving end (or, equivalently, the location of the notch along the body receiving end is said to correspond to the location of the protrusion along the sample receiving end). For example, in FIGS. 8A, 8C, distances D1a, D1c are approximately equal to D2a, D2c, respectively; and thus, the locations of protrusions 8010a, 8010c correspond to notches 8020a, 8020c, respectively.

In other embodiments, distance D1 and distance D2 are not equal or approximately equal, and the location of the protrusion along the sample receiving end is said to be offset from the location of the notch along the body receiving end (or, equivalently, the location of the notch along the body receiving end is offset from the location of the protrusion along the sample receiving end). For example, in FIG. 8B, distance D1b is not equal to D2b; and thus, the location of protrusion 8010b does not correspond to notch 8020b.

In some embodiments, the protrusion may be substantially in a center region or side region of the sample receiving end (i.e., the majority of the protrusion is in the center or side region). In some embodiments, the location of the notch may be substantially in a center region or side region of the body receiving end (i.e., the majority of the notch is in the center or side region). For example, in FIG. 8A, protrusion 8010a and notch 8020a are entirely in side region 8081. In FIG. 8B, protrusion 8010b is entirely in side region 8081 while notch 8020b is entirely in center region 8090. In FIG. 8C, protrusion 8010c and notch 8020c are substantially in side region 8081, although not entirely in side region 8081, because the majority of protrusion 8010c and notch 8020c are in side region 8081.

In some embodiments, the protrusion and/or notch is located entirely to one side of the bisecting line, in which case it does not extend to the bisecting line. In other embodiments, the protrusion and/or notch is located such that it extends to or beyond the bisecting line. For example, in FIG. 8A, protrusion 8010a and notch 8020b are located entirely to one side of bisecting line 8070. In FIG. 8B, protrusion 8010b is entirely to one side of bisecting line 8070 while notch 8020b is located such that it extends to both sides of the bisecting line 8070. In FIG. 8C, protrusion 8010c and notch 8020c are located such that they extend beyond the bisecting line 8070.

In some embodiments, the peak point of the protrusion and/or notch align within the width of the channel. Each peak point “aligns” where it perpendicularly crosses its base. For example, in FIGS. 8A-B, protrusions 8010a, 8010b and notches 8020a, 8020b align within channel 8030. In FIG. 8C, protrusion 8010c and notch 8020c do not align within channel 8030.

In some embodiments, the peak point of the protrusion and/or notch align to one side of the bisecting line of the channel. And, in some embodiments, the peak point of the protrusion and/or notch align on the bisecting line of the channel. For example, in FIG. 8A, peak points 8011a and 8021a align to one side of bisecting line 8070. In FIG. 8B, peak point 8011b aligns to one side of bisecting line 8070 while peak point 8021b aligns approximately on bisecting line 8070. In FIG. 8C, peak point 8011c and 8021c align to one side of bisecting line 8070.

In some embodiments, the peak point of the protrusion and/or notch align closer to a first side of the channel than to the bisecting line of the channel. For example, in FIG. 8A peak points 8011a and 8021a are closer to side 8040 of the channel than to bisecting line 8070, as shown by Y1a being less than Z1a, and Y2a being less than Z2a. In FIG. 8B peak point 8011b is closer to side 8040 than to bisecting line 8070, and 8021b is not closer to side 8040 of the channel than to bisecting line 8070, as shown by Y1b being less than Z1b, and Y2b being greater than Z2b (Z2b being approximately 0). In FIG. 8C, peak points 8011c,8021c are closer to side 8040 than to bisecting line 8070, as shown by Y1c being less than Z1c, and Y2c being less than Z2c.

It should be understood that the combinations of locations and shapes of the protrusion and notch may vary and are not limited to the specific embodiments shown in FIGS. 8A-8C.

It should also be understood that the number of protrusions on the sample receiving end may vary. For instance, in some embodiments, the sample receiving end of the test strip has only a single protrusion. In other embodiments, the sample receiving end has more than one protrusion.

It should also be understood that the number of notches on the body receiving end may vary. In some embodiments, the body receiving end of the test strip has only a single notch. In other embodiments, the body receiving end has more than one notch. In yet other embodiments, the body receiving end has no notch. For example, in some embodiments, the test strip may include one or more protrusions on the sample receiving end and no notch on the body receiving end. In embodiments where the body receiving end has no notch, a gap may be implemented between the body receiving end of the test strip and the lancet body to ensure that the channel is not blocked by the lancet body.

Method of Making a Plurality of Test Strips

As shown in FIGS. 4A-4B, the test strip is a layered structure comprising a base substrate 4006, cover substrate 4008, working electrode 4014, fill indication electrode 4822, assay chemistry, and supporting structures 4020, 4022 (e.g., adhesive strips) defining a channel 4024.

A method of making a plurality of test strips for use in an integrated lancet and test strip is provided. The method comprises forming a layered structure comprising a base substrate, a cover substrate, multiple electrodes, assay chemistry, and adhesive defining a plurality of channels. The method further comprises separating the layered structure into a plurality of test strips. Each test strip comprising multiple electrodes, at least one channel, a protrusion at a sample receiving end, and a notch at a body receiving end.

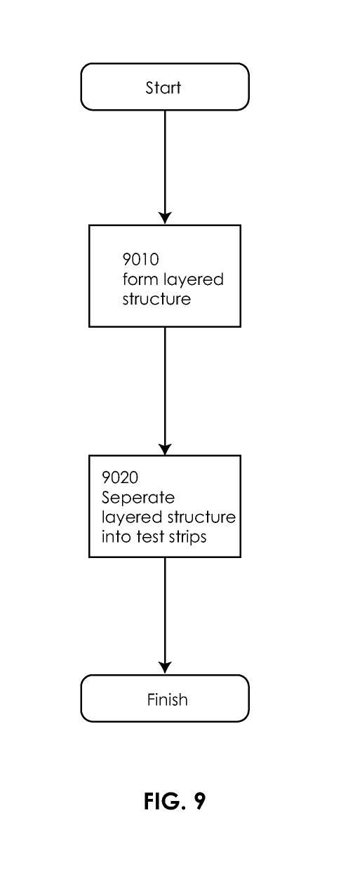

FIG. 9 illustrates a flow diagram for a method of making a plurality of test strips for use in an integrated lancet and testing meter, according to an embodiment. At block 9010, a layered structure is formed comprising a plurality of presensors which when separated form the test strips as described herein. The layered structure may comprise, for example, a base substrate, a cover substrate, a plurality of working electrodes, a plurality of counter and/or reference electrodes, fill-indicator electrodes, assay chemistry, and supporting structures (e.g., adhesive) defining a plurality of channels. In an embodiment, for example, a layer of electrically conductive material is provided on the base substrate. The layer of electrically conductive material may provide for a working electrode and counter and/or reference electrode of a plurality of presensors, for example. A layer of assay chemistry is also provided on the layered structure. A layer of non-conductive adhesive may then be applied on the base substrate, layer of electrically conductive material, and layer of assay chemistry. The layer of non-conductive adhesive may provide for the sides of the channel when the layered structure is completely formed. A cover substrate is then provided on all layers to form the layered structure of presensors.

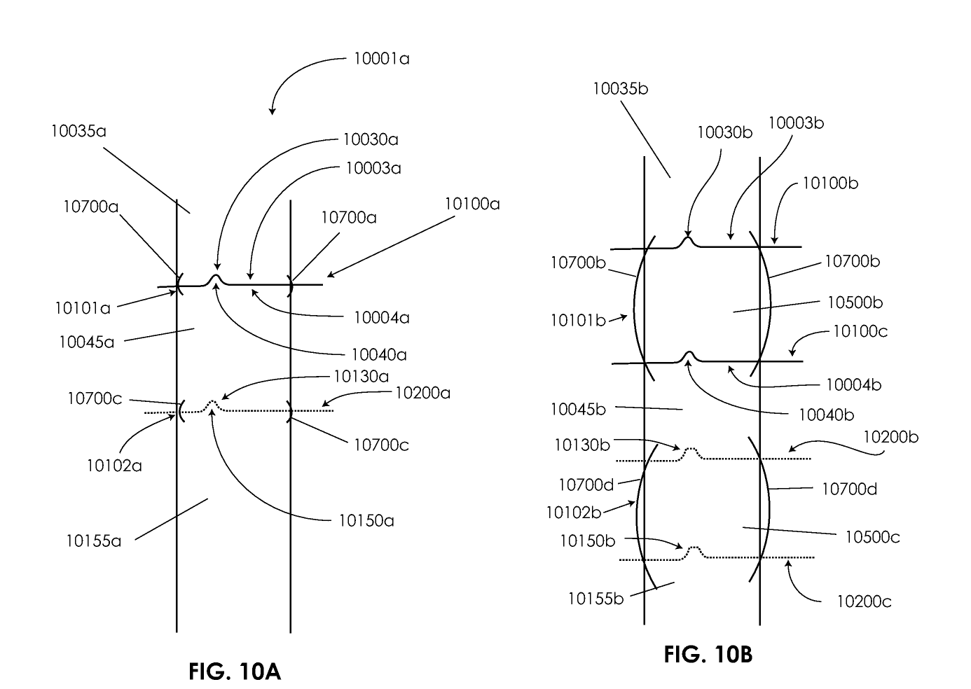

At block 9020, the layered structure is separated into a plurality of test strips. Each test strip may, for example, comprise at least one working electrode, at least one counter (or trigger) electrode, at least one channel, a protrusion at a sample receiving end, and a notch at a body receiving end of the test strip. Various methods may be implemented to separate the layered structure into a plurality of test strips. FIGS. 10A-B, 11A-B, and 12A-B provide details for separating the layered structure into a plurality of test strips, according to certain embodiments.

FIGS. 10A-B and 11A-B illustrate cutting the layered structure to form a plurality of test strips, according to certain embodiments. It should be understood that cutting also encompasses “punching” the layered structure—e.g., using two edges of a punching tool functioning as cutting blades. Each blade may have a contour comprising the shape of a protrusion/notch, so that when the blade makes a cut, the shape of the protrusion/notch is formed in the layered structure. Furthermore, each blade may have a contour comprising a shape of protrusion/notch independent of another blade and the shape of protrusion/notch defined by its contour. Thus, different shaped protrusions and/or notches may be formed in the layered structure. Still further, a blade may have a contour without a shape of a protrusion/notch, so that no protrusion/notch is formed within the layered structure by that blade. Still further, each blade may have a contour comprising the shapes of multiple protrusions/notches, so that when the blade makes a cut, the shapes of the multiple protrusions/notches are formed in the layered structure for that blade. When a blade has a contour comprising shapes of multiple protrusions/notches, the locations of the shapes of the protrusions/notches may vary depending on desired application. (It should be understood that the above applies equally when a perforated blade is being used).

In FIG. 10A, a cut 10100a (e.g., by a single blade) cuts the layered structure 10001a at a cutting site 10101a, as represented by parenthesis 11700a. On a first side 10003a of cutting site 10101a, a notch 10030a is formed on a first test strip 10035a. On a second side 10004a of the cutting site 10101a, a protrusion 10040a is formed on a second test strip 10045a. The second side 10004a is on an opposite side of the cutting site 10101a than the first side 10003a.

The next cut 10200a (e.g., represented by a dotted line at cutting site 10102a) will form the notch 10130a of the second test strip 10045a, and the protrusion 10150a of a third test strip 10155a. Cutting site 10102a is represented by parenthesis 10700c. This process is repeated as needed to obtain the desired number of test strips. It should be understood that the next cut may be positioned by either the cutting tool being moved, or by the layered structure 10001a being moved.

Cutting sites may be defined by two cuts from two different blades. For example, in FIG. 10B, a cutting site 10101b, as represented by parenthesis 10700b, is formed by two cuts—cut 10100b and cut 10100c. It should be understood that cut 10100b and cut 10100c may be executed simultaneously or sequentially by different blades of one or more cutting tools. Each blade may comprise similar or different shapes to provide similar or different shapes, respectively, for protrusions and notches.

Also, the portion of the layered structure within the cutting sites may either be disposable waste or test strips. Similarly, the portions of the layered structure between cutting sites may be disposable waste and/or test strips. Designs may vary based on application and as desired. For instance, if protrusions are to be substantially triangular and located in a side region while notches are to be substantially semicircular and located in a center region, then the first and second blades may be shaped accordingly.

FIG. 10B illustrates a cutting site 10101b, as represented by parenthesis 10700b, formed by two cuts—cut 10100b and cut 10100c—wherein a test strip is formed within each cutting site and between cutting sites.

On a first side 10003b of cutting site 10101b, a notch 10030b is formed on a first test strip 10035b. On a second side 10004b of the cutting site 10101b, a protrusion 10040b is formed on a second test strip 10045b. The second side 10004b is on an opposite side of the cutting site 10101b than the first side 10003b. And, as shown shaded, a third test strip 10500b is formed within the cutting site 10101b.

The next cuts 10200b,10200c (e.g., represented by dotted lines at cutting site 10102b), will form the notch 10130b of the second test strip 10045b, and the protrusion 10150b of a fourth test strip 10155b. Cutting site 10102b is represented by parenthesis 10700d. Fifth test strip 10500c is formed within cutting site 10102b and is also shown shaded. This process is repeated as needed to obtain the desired number of test strips. It should be understood that the next cut may be positioned by either the cutting tool(s) being moved, or by the layered structure 10001b being moved.

It should be noted that the shape of a blade may affect the shape of the corresponding protrusion and/or notch, as well as the location of the corresponding protrusion and/or notch. If the shapes of the two blades are different, then some test strips will have protrusions (and/or notches) with different shapes and/or locations than other test strips. For example, if the blade for cut 10100c was substantially rectangular instead of substantially triangular, then the notch for test strip 10500b and the protrusion for test strip 10045b would be substantially triangular.

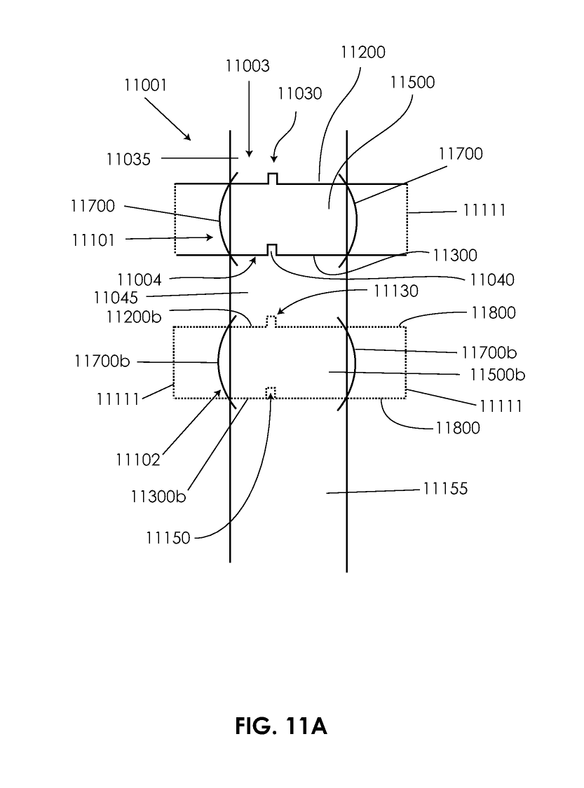

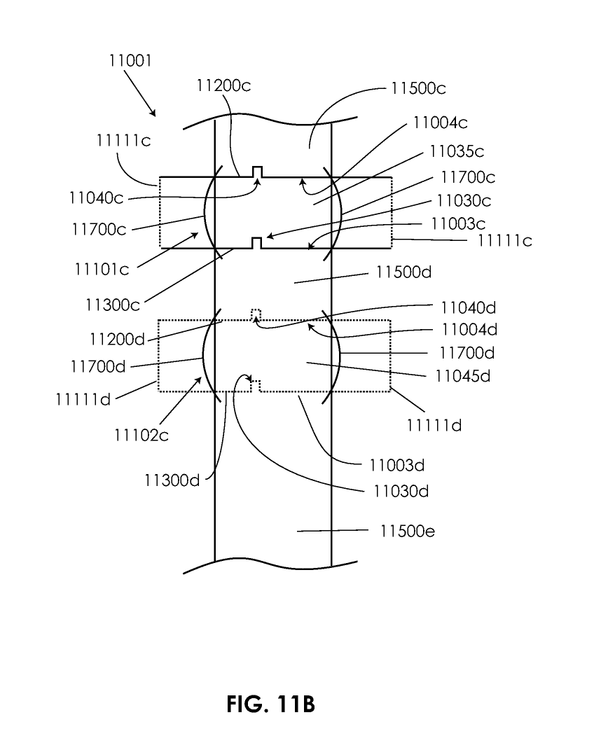

FIG. 11A illustrates cutting sites wherein a disposable piece is formed within each cutting site and test strips formed between cutting sites. As shown, cutting site 11101 is formed and defined by cutting out a disposable piece 11500 of layered structure 11001 at edges 11200 and 11300, as represented by parenthesis 11700. It should be understood that blade 11200 and blade 11300 may, for example, be edges on a single tool (as represented by dotted lines 11111) cutting out piece 11500, or two different blades on two different cutting tools cutting out piece 11500. In either case, the blades 11200 and 11300 define cutting site 11101. On a first side 11003 of cutting site 11101, a notch 11030 is formed on a first test strip 11035. On a second side 11004 of the cutting site 11101, a protrusion 11040 is formed on a second test strip 11045. The second side 11004 is on an opposite side of the cutting site 11101 than the first side 11003.

The next cut (e.g., at cutting site 11102, represented by dotted lines 11800) will form the notch 11130 of the second test strip 11045, and the protrusion 11150 of a third test strip 11155. Cutting site 11102 is formed and defined by cutting out a disposable piece 11500b of layered structure 11001 at blades 11200b and 11300b, as represented by parenthesis 11700b. Blades 11200b and 11300b define cutting site 11102. Disposable piece 11500b is formed within cutting site 11102. This process is repeated as needed to obtain the desired number of test strips. It should be understood that the next cut may be positioned by either the cutting tool(s) being moved, or by the layered structure 11001b being moved.

Alternatively, the disposable piece may be formed between cutting sites and test strips formed within the cutting sites, as illustrated in FIG. 11B. As shown, cutting site 11101c is formed and defined by cutting out a first test strip 11035c of layered structure 11001 at blades 11200c and 11300c, as represented by parenthesis 11700c.

It should also be understood that blade 11200c and blade 11300c may, for example, be edges on a single tool (as represented by dotted lines 11111c) cutting out test strip 11035c, or two different blades on two different cutting tools cutting out test strip 11035c.

In either case, the blades 11200c and 11300c define cutting site 11101c. On a first inside 11003c of cutting site 11101, a notch 11030c is formed on first test strip 11035c. On a second inside 11004c of the cutting site 11101c, a protrusion 11040c is formed on first test strip 11035c.

The next cut (e.g., at cutting site 11102d, represented by dotted blades 11200d and 11300d and parenthesis 11700d) will form the notch 11130d of the second test strip 11045d, and the protrusion 11150d of the second test strip 11145d. Cutting site 11102d is formed and defined by cutting out second test strip 11045d of layered structure 11001 at blades 11200d and 11300d, as represented by parenthesis 11700d. Second test strip 11145d is formed within cutting site 11102d. Disposable pieces 11500c, 11500d, and 11500e are formed between cutting sites. This process is repeated as needed to obtain the desired number of test strips. It should be understood that the next cut may be positioned by either the cutting tool(s) being moved, or by the layered structure 11001b being moved.

Again, it should be noted that the shape of each blade may affect the shape of the corresponding protrusion and/or notch, as well as the location of the corresponding protrusion and/or notch.

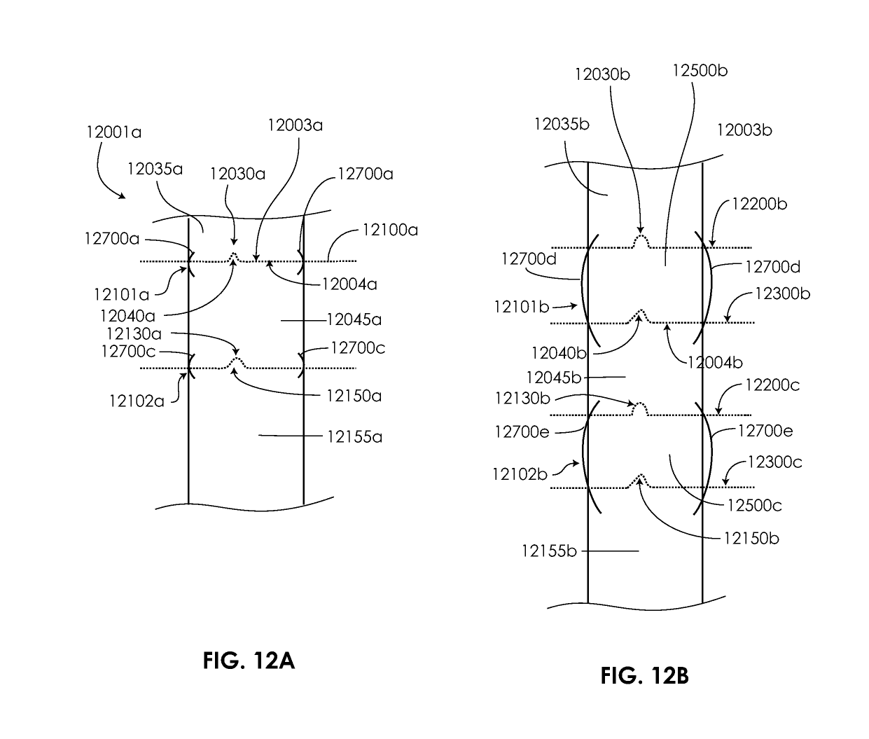

FIGS. 12A-12B illustrate perforating the layered structure to form a plurality of test strips after breaking along the perforation, according to certain embodiments.

In FIG. 12A, a perforation 12100a perforates (e.g., by a perforated blade) the layered structure 12001a at a perforation site 12101a, as represented by parenthesis 12700a. On a first side 12003a of perforation site 12101a, a notch 12030a is formed on a first test strip 12035a after breaking along the perforation. On a second side 12004a of the perforation site 12101a, a protrusion 12040a is formed on a second test strip 12045a after breaking along the perforation. The second side 12004a is on an opposite side of the perforation site 12101a than the first side 12003a.

The next perforation (e.g., represented by a dotted line at perforation site 12102a), will form the notch 12130a of the second test strip 12045a, and the protrusion 12150a of a third test strip 12155a. Perforation site 10102a is represented by parenthesis 10700c. This process is repeated as needed to obtain the desired number of test strips. It should be understood that the next perforation may be positioned by either the perforation tool being moved, or by the layered structure 12001a being moved. Furthermore, it should be understood that the breaking along one or more perforations may be performed at various times as so desired—e.g., breaking along the perforation after each perforation; breaking along the perforations after every two perforations (thus breaking off an individual test strip), breaking along all perforations after all perforation are performed on the layered structure; etc.

Perforation sites may be also be defined by two perforated cuts from two different perforated blades. It should be understood that perforations may be executed simultaneously or sequentially by different perforated blades of one or more cutting tools. The portion of the layered structure within the perforation sites may either be disposable waste or test strips. Similarly, the portions of the layered structure between perforation sites may be disposable waste and/or test strips. Designs may vary based on application and as desired. For instance, if protrusions are to be substantially triangular and located in a side region while notches are to be substantially semicircular and located in a center region, then the first and second perforated blades may be shaped accordingly.

It should be noted that the shape of a blade may affect the shape of the corresponding protrusion and/or notch, as well as the location of the corresponding protrusion and/or notch. If the shapes of the two blades are different, then some test strips will have protrusions (and/or notches) with different shapes and/or locations than other test strips.

FIG. 12B illustrates test strips being formed between perforation sites and disposable pieces being formed within each perforation site. As shown, perforations 12200b and 12300b define perforation site 12101b. It should be understood that perforations 12200b and 12300b may be executed by the same perforated blade or different perforated blades from different cutting tools. Perforations 12200b and 12300b define perforation site 12101b, as represented by parenthesis 12700d. On a first side 12003b of perforation site 12101b, a notch 12030b (shown in this example as substantially semi-circular) is formed on a first test strip 12035b after breaking along the perforation. On a second side 12004b of the perforation site 12101b, a protrusion 12040b (shown in this example as substantially triangular) is formed on a second test strip 12045b after breaking along the perforation. The second side 12004b is on an opposite side of the perforation site 12101b than the first side 12003b. As shown shaded, disposable pieces 12500b,12500c are formed within the perforation sites 12101b,12102b, respectively.

The next perforations 12200c, 12300c (e.g., represented by dotted lines at perforation site 12102b) will form the notch 12130b of the second test strip 12035b, and the protrusion 12150b of a third test strip 12155b. Perforation site 12102b is represented by parenthesis 12700e. Disposable piece 12500c is shown shaded and is formed within cutting site 12102b after breaking the perforations 12200c, 12300c. This process is repeated as needed to obtain the desired number of test strips. It should be understood that the next perforation may be positioned by either the perforation tool being moved, or by the layered structure 12001a being moved.

It should be understood that although not shown, test strips may be formed within the perforated sites. As similarly described for the cutting technique of FIG. 10B, it may be shown that tests strips may be formed both within perforation sites and between perforation sites. In such case, for example, the disposable pieces 12035b,12500c shown in FIG. 12B, would instead be test strips with a substantially semicircularly shaped protrusions and substantially triangularly shaped notches. Furthermore, as similarly described for the cutting technique of FIG. 11B, it may be shown that test strips may be formed within perforation sites while disposable pieces are formed between perforation sites. In such case, for example, test strips 12035b,12045b,12155b shown in FIG. 12B, would instead be disposable pieces; and disposable pieces 12500b,12500c shown in FIG. 12B, would instead be test strips.

The earlier-mentioned figures and description pertaining to shapes and locations of protrusions and notches are still applicable. Although specific protrusion shapes and locations are shown in the specific embodiments of FIGS. 10A-10B, 11A-B, and 12A-12B, it should be understood that the combination of shapes and locations of the protrusion and notch may vary and are not limited to the specific embodiments shown.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. An integrated lancet and test strip for measuring an analyte level, comprising:
a body comprising a test strip receiving end and a lancet end;
a lancet needle coupled to the lancet end of the body; and
a test strip coupled to the test strip receiving end of the body, wherein the test strip comprises:
multiple electrodes and assay chemistry for determining an analyte level in a sample;
a protrusion at a sample receiving end of the test strip; and
a body receiving end of the test strip;
wherein the test strip and lancet needle are configured such that the test strip is not in fluid communication with the lancet needle.

2. The integrated lancet and test strip of claim 1, wherein the test strip further comprises a notch at the body receiving end of the test strip, and wherein the shape of the notch and protrusion are substantially triangular.

3. The integrated lancet and test strip of claim 1, wherein the test strip further comprises a notch at the body receiving end of the test strip, and wherein the notch comprises a shape different than a shape of the protrusion at the sample receiving end of the test strip.

4. The integrated lancet and test strip of claim 1, wherein the test strip further comprises a notch at the body receiving end of the test strip, and wherein the notch comprises a shape substantially the same as a shape of the protrusion at the sample receiving end of the test strip.

5. The integrated lancet and test strip of claim 4, wherein the test strip further comprises a notch at the body receiving end of the test strip, and wherein the shape of the notch and protrusion are one selected from the group consisting of substantially triangular, substantially semi-circular, and substantially rectangular.

6. The integrated lancet and test strip of claim 1, wherein the body receiving end of the test strip is opposite the fluid receiving end of the test strip.

7. The integrated lancet and test strip of claim 1, wherein the test strip further comprises a notch at the body receiving end of the test strip, and wherein a location of the notch along the body receiving end corresponds to a location of the protrusion along the sample receiving end.

8. The integrated lancet and test strip of claim 7, wherein the test strip further comprises a notch at the body receiving end of the test strip, and wherein the location of the notch is in a side region of the body receiving end, and the location of the protrusion is in a side region of the sample receiving end.

9. The integrated lancet and test strip of claim 1, wherein the test strip further comprises a notch at the body receiving end of the test strip, and wherein a location of the notch along the body receiving end is offset from a location of the protrusion along the sample receiving end.

10. The integrated lancet and test strip of claim 9, wherein the test strip further comprises a notch at the body receiving end of the test strip, and wherein the location of the notch is in a center region of the body receiving end, and the location of the protrusion is outside of a center region of the sample receiving end.

11. The integrated lancet and test strip of claim 1, wherein the test strip further comprises a channel extending from the sample receiving end to the body receiving end of the test strip, wherein the protrusion is located entirely on one side of a bisecting line of the channel.

12. The integrated lancet and test strip of claim 1, wherein the test strip further comprises:
a notch at the body receiving end of the test strip; and
a channel extending from the sample receiving end to the body receiving end of the test strip, wherein a peak point of the protrusion and a peak point of the notch align within a width of the channel.

13. The integrated lancet and test strip of claim 12, wherein the peak point of the protrusion aligns to one side of a bisecting line of the channel.

14. The integrated lancet and test strip of claim 13, wherein the protrusion comprises a substantially triangular shape.

15. The integrated lancet and test strip of claim 13, wherein the test strip further comprises a notch at the body receiving end of the test strip, and wherein the peak point of the notch aligns on the bisecting line of the channel.

16. The integrated lancet and test strip of claim 13, wherein the peak point of the protrusion aligns closer to a first side of the channel than to the bisecting line of the channel.

17. The integrated lancet and test strip of claim 13, wherein the test strip further comprises:
a first and second adhesive strip extending on a first and second side of the channel, respectively, and defining the channel.

18. The integrated lancet and test strip of claim 17, wherein the peak point of the protrusion aligns closer to the first adhesive strip than to the bisecting line of the channel.

19. The integrated lancet and test strip of claim 17, wherein a shape of the protrusion is one selected from the group consisting of substantially triangular, substantially semi-circular, and substantially rectangular.

20. The integrated lancet and test strip of claim 17, wherein the test strip further comprises a notch at the body receiving end of the test strip, and wherein the peak point of the notch aligns on the bisecting line of the channel.

21. The integrated lancet and test strip of claim 1, wherein the test strip further comprises:
a channel extending from the sample receiving end to the body receiving end of the test strip, the channel defined by a first and second structure on each side of the channel, the first structure providing structural rigidity to the protrusion.

22. The integrated lancet and test strip of claim 21, wherein the first and second structure are adhesive strips.

23. The integrated lancet and test strip of claim 1, wherein at least one of the multiple electrodes comprises an analyte response enzyme.

24. The integrated lancet and test strip of claim 23, wherein the analyte response enzyme is a glucose response enzyme or a ketone responsive enzyme.

25-103. (canceled)
